(19) United States
(12) Patent Application Publication
(10) Pub. No.: US 2003/0072725 A1
(43) Pub. Date: Apr. 17, 2003

(54) Topical cosmetic agents containing 2-hydrazino-1,3 -heteroazoles

(76) Inventors: Jakob Peter Ley, Holzminden (DE); William Johncock, Hoxter (DE); Roland Langer, Bevern (DE)

Correspondence Address: BAYER CORPORATION, PATENT DEPARTMENT, 100 BAYER ROAD, PITTSBURGH, PA, 15205, US

(21) Appl. No.: 10/204,517

(22) PCT Filed: Feb. 13, 2001
(86) PCT No.: PCT/EP01/01563

(30) Foreign Application Priority Data
Feb. 25, 2000 (DE) 08907.0100

(51) Int. Cl. A61K 7/42

(52) U.S. Cl. 424/59

(57) ABSTRACT

The inventive 2-hydrazino-1,3-heteroazoles or salts thereof are, as very good tyrosinase inhibitors, active constituents in topical cosmetic agents, especially in cosmetic or dermatological skin brightening agents.

The invention relates to topical cosmetic compositions, in particular cosmetic or dermatological skin-lightening compositions, comprising 2-hydrazino-1,3-heteroazoles, for cosmetic or dermatological applications.

Skin lightening active ingredients usually interfere with melanin metabolism or catabolism. The melanins, which are usually brown to black in color, are formed in the melanocytes of the skin, transferred to the keratinocytes and cause the coloration of skin or hair. The brown-black eumelanins are formed in mammals predominantly from hydroxy-substituted aromatic amino acids such as L-tyrosine and L-DOPA, and the yellow to red pheomelanins are additionally formed from sulfur-containing molecules (Cosmetics& Toiletries 1996, 111 (5), 43-51). Starting from L-tyrosine, the copper-containing key enzyme tyrosinase forms L-3,4-dihydroxyphenylalanine (L-DOPA), which for its part is oxidized again by the tyrosinase via the red-brown dopaquinone to give melanin. A comparison of tyrosinases from plants, fungi and mammal cells shows that the mechanism and the substrate specificity is comparable in all of the tyrosinases investigated.

If, for some reason, the melanin-forming melanocytes are not distributed evenly in the human skin, pigmentation spots form, which are either lighter or darker than the surrounding areas of skin. In order to overcome this problem, skin-lightening compositions are offered on the market which help to at least partially even out pigmentation spots. In addition, many people have a desire to lighten their naturally dark skin color. Very safe and effective skin-lightening compositions are required for this purpose. Many skin-lightening compositions comprise tyrosinase inhibitors of greater or lesser strength.

In commercially available skin-lightening compositions hydroquinone, hydroquinone derivatives, such as, for example, arbutin, vitamin C, derivatives of ascorbic acid, such as, for example, ascorbyl palmitate, kojic acid and derivatives of kojic acid, such as, for example, kojic acid dipalmitate in particular are used (Cosmetics & Toiletries 1996, 111(5), 43-51).

One of the most frequently used skin lighteners is hydroquinone. However, the substance has a cytotoxic effect toward melanocytes and can damage the skin. For this reason, such preparations are no longer authorized for cosmetic applications in, for example, Japan and South Africa. In addition, hydroquinone is very oxidation-sensitive and can only be stabilized with difficulty in cosmetic formulations.

Vitamin C and ascorbic acid derivatives have only an inadequate action on the skin. Further, they do not act directly as tyrosinase inhibitors, but reduce the colored intermediates of melanin biosynthesis.

Kojic acid (5-hydroxy-2-hydroxymethyl-4-pyranone) is a tyrosinase inhibitor which, by chelating the copper atoms of the enzyme, inhibits the catalytic activity of the latter; it is used in commercial skin-lightening compositions. The substance is formed predominantly in Aspergillus cultures and can only be isolated therefrom in small amounts. In addition, kojic acid is not stable in aqueous solutions and is thus unsuitable for most cosmetic compositions (Cosmetics & Toiletries 1999, 9, p. 27).

The object of the present invention was to find topical cosmetic compositions, in particular cosmetic or dermatological skin-lightening compositions, which comprise inexpensive highly effective tyrosinase inhibitors which are easy to prepare.

The invention thus provides topical cosmetic compositions comprising 2-hydrazino-1,3-heteroazoles of the general formula

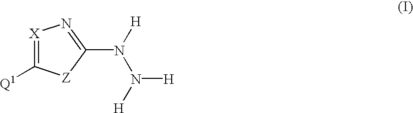

or salts thereof,

where

Z is a sulfur or an oxygen atom or —NH,

and

X is a nitrogen atom or a carbon atom substituted with Q², C—Q²,

and

either

Q¹and Q², independently of one another, are hydrogen atoms, optionally hydroxyl- or alkyloxy-substituted unbranched, branched or cyclic alkyl, alkenyl, 1-oxoalkyl or 1-oxoalkenyl groups having 1 to 18 carbon atoms,

optionally substituted aryl groups having 6 to 15 carbon atoms, optionally substituted heterocyclyl groups having 2 to 15 carbon atoms and at least one heteroatom chosen from the group oxygen, nitrogen or sulfur, optionally substituted arylalkyl or aryl-1-oxoalkyl groups of 7 to 16 carbon atoms,

halogen atoms, nitro groups or

groups —COOR⁵, —OR⁵, —NR⁵R⁶, —SO₂OR⁵, —SO₂NR⁵R⁶ or

—PO(OR⁵)(OR⁶),

or

Q¹and Q² together are a radical of the general formula (II)

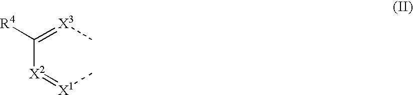

where

X¹, X² and X³, independently of one another, are either nitrogen atoms or carbon atoms with the radicals R¹, R² or R³, respectively,

and

R¹, R², R³ and R⁴, independently of one another, are hydrogen atoms, optionally hydroxyl- or alkyloxy-substituted unbranched, branched or cyclic alkyl, alkenyl, 1-oxoalkyl or 1-oxoalkenyl groups having 1 to 18 carbon atoms,

optionally substituted aryl groups having 6 to 15 carbon atoms, optionally substituted heterocyclyl groups having 2 to 15 carbon atoms and at least one heteroatom chosen from the group oxygen, nitrogen or sulfur,

optionally substituted arylalkyl or aryl-1-oxoalkyl groups of 7 to 16 carbon atoms,

halogen atoms, nitro groups or

groups —COOR⁵, —OR⁵, —NR⁵R⁶, —SO₂OR⁵, —SO₂NR⁵R⁶ or

—PO(OR⁵)(OR⁶)

and

R⁵ and R⁶ independently of one another, are hydrogen atoms, optionally hydroxyl- or alkyloxy-substituted unbranched, branched or cyclic alkyl, alkenyl, 1-oxoalkyl or 1-oxoalkenyl groups having 1 to 18 carbon atoms, optionally substituted aryl groups having 6 to 15 carbon atoms,

optionally substituted arylalkyl or aryl-1-oxoalkyl groups of 7 to 16 carbon atoms.

The 2-hydrazino-1,3-heteroazoles according to the invention can also be in the form of their tautomers.

An unbranched, branched or cyclic alkyl group can contain 1 to 18, preferably 1 to 8, particularly preferably 1 to 6, carbon atoms. Examples which may be mentioned are: methyl, ethyl, 1-propyl, 2-propyl, 1-butyl, 2-butyl, tert-butyl, 2-methyl, 2-methylprop-1-yl, cyclopropyl, cyclopropylmethyl, 2,2-dimethylcyclopropyl, cyclobutyl, cyclopentyl, cyclohexyl and various positional isomers of methylpentyl.

An unbranched, branched or cyclic alkenyl group can contain 2 to 18, preferably 2 to 8, particularly preferably 2 to 6, carbon atoms. Examples which may be mentioned are: ethenyl, 1- or 2-propenyl, 1-, 2- or 3-butenyl, 2-methyl-1-propenyl, 2-methyl-2-propenyl, 1,3-butadienyl, 1,3-pentadienyl, 1,4-pentenyl, 2,4-pentenyl, the respective various straight-chain, cyclic or branched isomers of the pentenyl, hexenyl radicals. Particular preference is given to ethenyl, 1- or 2-propenyl, 1-, 2- or 3-butenyl, 2-methyl-1-propenyl, 2-methyl-2-propenyl, 3-methyl-1-pentenyl, 3-methyl-2-pentenyl, 3-methyl-3-pentenyl, cyclopentenyl, cyclopentadienyl, cyclohexadienyl and cyclohexenyl.

An unbranched, branched or cyclic 1-oxoalkyl group can contain 1 to 18, preferably 1 to 8, particularly preferably 1 to 5, carbon atoms. Examples which may be mentioned are: formyl, acetyl, propionyl, 2-methylpropionyl, butanoyl, 2-methylbutanoyl, 3-methylbutanoyl, 2,2-dimethylpropionyl, cyclopropylcarboxyl.

An unbranched, branched or cyclic 1-oxoalkenyl group can contain 3 to 18, preferably 3 to 8, particularly preferably 3 to 5, carbon atoms. Examples which may be mentioned are: prop-2-enoyl, 2-methylprop-2-enoyl, 2-ethylprop-2-enoyl, E- or Z-2-butenoyl, 3-butenoyl, E- or Z-2-methylbut-2-enoyl, E- or Z-3-methylbut-2-enoyl, Z- or E-2-pentenoyl, Z- or E-3-pentenoyl.

Aryl groups with 6 to 15 carbon atoms, preferably 6 to 10 carbon atoms may, for example, be: phenyl and naphthyl.

A heterocyclyl group having 2 to 15 carbon atoms and at least one atom from the group oxygen, sulfur or nitrogen in the ring generally consists of 1 to 3, preferably 1 or 2, five- or six-membered rings. The heterocyclyl group preferably contains 1 to 4, particularly preferably 1 or 2, heteroatoms. Preference is given to furan, pyrrole, thiophene, indole, isoindole, benzofuran, isobenzofuran, benzothiophene, isobenzothiophene, pyrazole, imidazole, 1,3- or 1,2-oxazole, 1,3- or 1,2-thiazole, 1,3-or 1,2-benzimidazole, 1,3- or 1,2-benzoxazole, 1,3- or 1,2-benzothiazole, pyridine, pyrimidine, pyrazine, 1,2-, 1,3- or 1,4-oxazine, 1,2-, 1,3- or 1,4-thiazine, quinoline, isoquinoline, benzo-1,2-, -1,3- or -1,4-diazine or partially or completely saturated derivatives thereof, e.g. tetrahydrofuran, 1,3-dioxolane, pyrrolidine, pyrroline, 1,3- or 1,4-dioxane, piperidine, tetrahydro-2H-pyrane, piperazine, oxirane or aziridine. Particular preference is given to furan, pyrrole, indole, imidazole, 1,3-thiazole, 1,3-benzothiazole, pyridine, pyrimidine, quinoline, isoquinoline or partially or completely saturated derivatives thereof, e.g. tetrahydrofuran, 1,3-dioxolane, pyrrolidine, 1,3- or 1,4-dioxane, piperidine or tetrahydro-2H-pyrane.

An arylalkyl group can consist of 6 to 15 carbon atoms, preferably of 7 to 8 carbon atoms and may, for example, be: benzyl, 2- or 1-phenylethyl.

An aryl-1-oxoalkyl group can consist of 6 to 15 carbon atoms, preferably of 7 to 8 carbon atoms and may, for example, be: benzoyl, phenylacetyl.

Substituents of the aryl, arylalkyl, aryl-1-oxoalkyl and heterocyclyl groups may, for example, be: hydrogen atoms, alkyl, hydroxyl, alkyloxy, thio, alkylthio, amino, alkylamino, dialkylamino, nitro, iodine, bromine, fluorine, chlorine, azido, thiocyanato, isothiocyanato, cyanato, isocyanato, nitrile, isonitrile, phosphate, alkylphosphate, dialkylphosphate, sulfonic acid, alkylsulfonate, sulfonamide, dialkylsulfonamide or alkylsulfonamide radicals. Particular preference is given to hydrogen atoms, alkyl, hydroxyl, alkyloxy, amino, dialkylamino, bromine, fluorine, chlorine, nitrile, sulfonic acid, sulfonamide or alkylsulfonate radicals.

The 2-hydrazino-1,3-benzoheteroazoles according to the invention are preferably used as cosmetic or dermatological skin lightening agents.

Preference is given to topical cosmetic compositions comprising 2-hydrazino-1,3-benzoheteroazoles of the general formula (I)

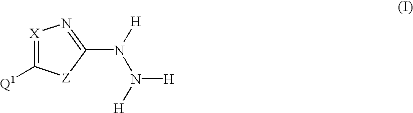

or salts thereof,

where

Z is a sulfur or oxygen atom or —NH,

and

X is a nitrogen atom or a carbon atom substituted by Q², C—Q²,

and

either

Q¹and Q², independently of one another, are hydrogen atoms, methyl, trifluoromethyl, ethyl, tert-butyl, allyl, 3-methylbut-2-en-1-yl or acetyl groups, chlorine or fluorine atoms, nitro groups, groups —COOR⁵, —OR⁵, —NR⁵R⁶, —SO₂OR⁵, —SO₂NR⁵R⁶, —PO(OR⁵)(OR⁶), phenyl, pyridyl, pyrazinyl groups optionally substituted on the aromatic by alkyl, hydroxyl, alkyloxy, amino, dialkylamino, bromine, fluorine, chlorine, nitrile, sulfonic acid, sulfonamide or alkylsulfonate radicals, phenylmethyl or benzoyl groups,

or

Q¹and Q² together are a radical of the general formula (II)

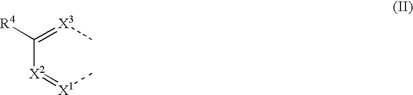

where

x¹, x² and X³ are carbon atoms having the radicals R¹, R² and R³, respectively,

and

R¹, R², R³ and R⁴, independently of one another, are hydrogen atoms, methyl, trifluoromethyl, ethyl, tert-butyl, allyl, 3-methylbut-2-en-1-yl or acetyl groups, phenyl, pyridyl, pyrazinyl groups, phenylmethyl, 4-methylphenylmethyl, 4-methoxyphenylmethyl or benzoyl groups, chlorine or fluorine atoms, nitro groups, groups —COOR⁵, —OR⁵, —NR⁵R⁶, —SO₂OR⁵, —SO₂NR⁵R⁶or —PO(OR⁵)(OR⁶)

and

R⁵ and R⁶, independently of one another, are hydrogen atoms, methyl, trifluoromethyl, ethyl, tert-butyl, allyl, 3-methylbut-2-en-1-yl, acetyl, propionyl or pivaloyl groups, phenyl, pyridyl, pyrazinyl groups, phenylmethyl, 4-methylphenylmethyl, 4-methoxyphenylmethyl or benzoyl groups.

Particular preference is given to topical cosmetic compositions which comprise, as active ingredient,

2-hydrazino-1,3-benzothiazole

5,6-dimethoxy-2-hydrazino-1,3-benzothiazole

6-methoxy-2-hydrazino-1,3-benzothiazole

2-hydrazino-1,3-benzothiazole-5-sulfonic acid

2-hydrazino-1,3-benzothiazole-6-sulfonic acid

6-tert-butyl-2-hydrazino-1,3-benzothiazole

6-methyl-2-hydrazino-1,3-benzothiazole

2-hydrazinothiazolo[5,4-b]pyridine

2-hydrazino-1,3-benzoxazole

2-hydrazino-1H-benzimidazole

2-hydrazino-5-(4-fluorophenyl)-1,3,4-thiadiazole

2-hydrazino-4-phenyl-1,3-thiazole

2-hydrazino-4-methyl-1,3-thiazole hydrochloride.

The 2-hydrazino-1,3-thiazoles, some of which are known, can be prepared, for example, in accordance with the process described in Organic Preparations and Procedures Int. 1974, 6(4), 179-182 from the 2-amino-1,3-thiazoles. The latter can, for example, be synthesized from the corresponding anilines or heterocyclic amines by reaction with inorganic thiocyanate salts and subsequent oxidative ring closure of the N-arylthiourea (J. Indian Chem. Soc. 1989, 66, 39-41). The 2-hydrazino-1,3-oxazoles according to the invention can, for example, be synthesized in accordance with the process described in J. Amer. Chem. Soc. 1953, 75, 712. A preparation process for the 2-hydrazino-1,3-imidazoles according to the invention has been described, for example, in DE 614,327.

The 2-hydrazino-1,3-benzothiazoles according to the invention can be synthesized, for example, starting from an optionally substituted aniline by reaction with potassium, sodium or ammonium thiocyanate, then by chlorine-, bromine- or iodine-mediated oxidative ring closure and finally by reaction with hydrazine or hydrazine hydrate.

The preparation can be explained by reference to the illustrative example 2-hydrazino-6-tert-butyl-1,3-benzothiazoles by the following equation:

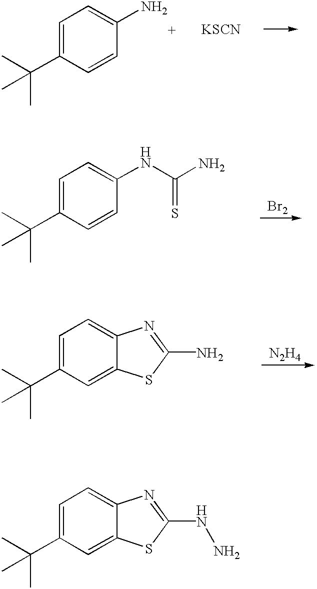

Surprisingly, we have now found that the 2-hydrazino-1,3-heteroazoles present in the topical cosmetic compositions according to the invention are particularly effective tyrosinase inhibitors. In particular, many of the 2-hydrazino-1,3-heteroazoles according to the invention have an effectiveness which is either comparable or better than that of kojic acid. The novel substituted and benzo-fused 2-hydrazino-1,3-thiazoles or the substituted 2-hydrazino-1,3,4-thiadiazoles are particularly suitable. They can thus be used as active ingredients in cosmetic or dermatological skin-lightening compositions.

The topical cosmetic compositions according to the invention, in particular the cosmetic or dermatological skin-lightening compositions, comprising the 2-hydrazino-1,3-heteroazoles are prepared by customary methods known per se by incorporating one or more of the novel 2-hydrazino-1,3-heteroazoles of the general formula I or salts thereof into cosmetic or dermatological formulations which have the customary composition and, in addition to the skin lightening action, can also serve for the treatment, the protection, the care and the cleansing of the skin and/or the hair and as make-up products in decorative cosmetics.

The topical cosmetic compositions according to the invention comprise 2-hydrazino-1,3-heteroazoles in an effective amount and optionally other constituents. They comprise 0.001% by weight to 30% by weight, preferably 0.001 to 20% by weight, but in particular 0.001% by weight to 5% by weight, based on the total weight of the formulation, of the 2-hydrazino-1,3-heteroazoles and can be in the form of water-in-oil, oil-in-water, water-in-oil-in-water or oil-in-water-in-oil emulsions, microemulsions, gels, solutions e.g. in oils, alcohols or silicone oils, as sticks, as aerosols, sprays and also foams. Further customary cosmetic auxiliaries and additives may be present in amounts of from 5 to 99.999% by weight, preferably 10 to 80% by weight, based on the total weight of the formulation. In addition, the formulations can have water in an amount up to 99.999% by weight, preferably 5 to 80% by weight, based on the total weight of the formulation.

To prepare the topical cosmetic compositions according to the invention, in particular the cosmetic and dermatological skin-lightening compositions, in a further embodiment, the 2-hydrazino-1,3-heteroazoles of the general formula I may also be incorporated beforehand in liposomes, e.g. starting from phosphatidylcholine, in microspheres, in nanospheres or else in capsules made of a suitable matrix, e.g. made of natural or synthetic waxes, for example beeswax, carnauba wax, silicone wax or stearyl alcohol, eicosanol, cetyl alcohol, stearine or paraffin wax or made of gelatins.

The topical cosmetic compositions according to the invention, in particular the cosmetic and dermatological skin-lightening compositions, can comprise cosmetic auxiliaries and additives as are customarily used in such preparations, e.g. sunscreens (e.g. organic or inorganic light filter substances, preferably micropigments), preservatives, bactericides, fungicides, virucides, cooling agents, plant extracts, antiinflammatory active ingredients, substances which accelerate wound healing (e.g. chitin or chitosan and derivatives thereof), film-forming substances (e.g. polyvinylpyrrolidones or chitosan or derivatives thereof), customary antioxidants, vitamins (e.g. vitamin C and derivatives, tocopherols and derivatives, vitamin A and derivatives), 2-hydroxycarboxylic acids (e.g. citric acid, malic acid, L-, D- or di-lactic acid), perfumes, antifoams, dyes, pigments which have a coloring action, thickeners, surface-active substances, emulsifiers, emollients, humectants and/or moisturizers (e.g. glycerol or urea), fats, oils, unsaturated fatty acids or derivatives thereof (e.g. linoleic acid, α-linolenic acid, γ-linolenic acid or arachidic acid and the natural or synthetic esters thereof in each case), waxes or other customary constituents of a cosmetic or dermatological formulation, such as alcohols, polyols, polymers, foam stabilizers, electrolytes, organic solvents, silicone derivatives or chelating agents (e.g. ethylenediaminetetraacetic acid and derivatives).

The amounts of amounts of cosmetic or dermatological auxiliaries and additives and perfume to be used in each case can easily be determined by simple exploratory experiments by the person skilled in the art, depending on the nature of the product in question.

For use, the topical cosmetic compositions according to the invention, in particular the cosmetic or dermatological skin-lightening compositions, comprising 2-hydrazino-1,3-heteroazoles of the general formula I are applied to the skin and/or the hair in a sufficient amount in the manner customary for cosmetics.

Preferably, the topical cosmetic compositions according to the invention comprising 2-hydrazino-1,3-heteroazoles of the formula I or salts thereof can also comprise other active ingredients for skin lightening. In particular, the topical cosmetic compositions according to the invention can also comprise benzaldoximes having at least one aromatic hydroxyl or alkoxy group, kojic acid, kojic acid derivatives, ascorbic acid, ascorbic acid derivatives, hydroquinone, hydroquinone derivatives, sulfur-containing molecules (e.g. glutathione or cystein) or other synthetic or natural active ingredients for skin lightening, it being possible to use the latter also in the form of a plant extract (e.g. tocopherols and derivatives, arbutin (e.g. from bearberry extract), aloesin (e.g. from aloe extract), grapefruit extract and rice extract).

The amount of the abovementioned other active ingredients for skin lightening, given by way of example (one or more compounds), which are not identical to the 2-hydrazino-1,3-heteroazoles present in the topical cosmetic compositions according to the invention, in the skin-lightening compositions according to the invention can be 0.001 to 30% by weight, preferably 0.001 to 20% by weight, particularly preferably 0.001 to 5% by weight, based on the total weight of the preparation.

Particular preference is given to those topical cosmetic compositions, in particular cosmetic and dermatological skin-lightening compositions, which are also in the form of a sunscreen. In addition to an effective amount of the 2-hydrazino-1,3-heteroazoles of the general formula I, these also comprise sunscreen substances, preferably organic or inorganic light filter substances, in particular micropigments. The skin-lightening compositions according to the invention can, however, also comprise UVA and/or UVB filter substances, where the total amount of filter substances can be 0.1 to 30% by weight, preferably 0.5 to 10% by weight, based on the total weight of the preparations, giving sunscreens for skin and hair. Examples of UV filter substances which can be used are 3-benzylidenecamphor derivatives (e.g. 3-(4-methylbenzylidene)-dl-camphor), aminobenzoic acid derivatives (e.g. 2-ethylhexyl 4-(N,N-dimethylamino)benzoate or menthyl anthranilate), 4-methoxy-cinnamates (e.g. 2-ethylhexyl p-methoxycinnamate or isoamyl p-methoxycinnamate), benzophenones (e.g. 2-hydroxy-4-methoxybenzophenone), mono- or polysulfonated UV filters [e.g. 2-phenylbenzimidazole-5-sulfonic acid, sulisobenzones or 1,4-bis(benzimidazolyl)-benzene-4,4′, 6,6′-tetrasulfonic acid and 3,3′-(1,4-phenylenedimethylidene)-bis-(7,7-dimethyl-2-oxo-bicyclo-[2,2,1]heptane-1-methanesulfonic acid) and salts thereof], salicylates (e.g. 2-ethylhexyl salicylate or homomenthyl salicylate), triazines {e.g. 2,4-bis-[4-(2-ethylhexyloxy)-2-hydroxy-phenyl]-6-(4-methoxyphenyl)-1,3,5-triazine, bis-(2-ethylhexyl) 4,4′-([6-([(1,1-dimethylethyl) aminocarbonyl]phenylamino)-1,3,5-triazin-2,4-diyl]diimino)-bisbenzoate, 2-cyanopropenoic acid derivatives (e.g. 2-ethylhexyl 2-cyano-3,3-diphenyl-2-propenoate), dibenzoyl derivatives (e.g. 4-tert-butyl-4′-methoxy-dibenzoylmethane), polymer-bonded UV filters (e.g. polymers of N-[2-(or 4)-(2-oxo-3-bornylidene)methyl]benzylacrylamide) or pigments (e.g. titanium dioxides, zirconium dioxides, iron oxides, silicon dioxides, manganese oxides, aluminum oxides, cerium oxides or zinc oxides).

In a further preferred embodiment of the invention antioxidants or free-radical scavengers are also present in the topical cosmetic compositions comprising 2-hydrazino-1,3-heteroazoles of the general formula I or salts thereof. According to the invention, favorable antioxidants which may be used are all antioxidants which are customary or suitable for cosmetic and/or dermatological applications. The antioxidants are advantageously chosen from the group consisting of amino acids (e.g. glycine, histidine, 3,4-dihydroxyphenylalanine, tyrosine, tryptophan) and derivatives thereof, imidazoles (e.g. urocaninic acid) and derivatives thereof, peptides (D,L-carnosine, D-carnosine, L-carnosine, anserine) and derivatives thereof, carotenoids, carotenes (e.g. β-carotene, α-carotene, lycopene) and derivatives thereof, chlorogenic acid and derivatives thereof, lipoic acid and derivatives thereof, aurothioglucose, propylthiouracile and other thiols (e.g. thio-redoxin, glutathione, cysteine, cystine, cystamine and the glycosyl and N-acyl derivatives thereof or alkylesters thereof), and salts thereof, dilauryl thiodipropionate, distearyl thiodipropionate, thiodipropionic acid and derivatives thereof, and also phenolic acid amides of phenolic benzylamines (e.g. homovanillic acid amides, 3,4-dihydroxyphenylacetic acid amides, ferulic acid amides, sinapic acid amides, caffeic acid amides, dihydroferulic acid amides, dihydrocaffeic acid amides, vanillomandelic acid amides or 3,4-dihydroxymandelic acid amides of 3,4-dihydroxybenzylamine, 2,3,4-trihydroxybenzylamine or 3,4,5-trihydroxybenzylamine), catechol oximes or catechol oxime ethers (e.g. 3,4-dihydroxybenzaldoxime or 3,4-dihydroxybenzaldehyde O-ethyloxime), and also (metal) chelating agents (e.g. 2-hydroxy fatty acids, phytic acid, lactoferin), humic acid, bile acids, bile extracts, bilirubin, biliverdin, folic acid and derivatives thereof, ubiquinone and ubiquinol and derivatives thereof, vitamin C and derivatives thereof (e.g. ascorbyl palmitate, magnesium ascorbyl phosphate, ascorbyl acetate), tocophe-roles and derivatives (e.g. vitamin E acetate), vitamin A and derivatives (e.g. vitamin A palmitate), rutinic acid and derivatives thereof, flavonoids (e.g. quercetin, glucosylrutin) and derivatives thereof, phenolic acids (e.g. gallic acid, ferulic acid) and derivatives thereof (e.g. propyl gallate, ethyl gallate, octyl gallate), furfurylideneglucitol, butylhydroxytoluene, butylhydroxyanisole, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, resveratrol) and the derivatives of these said active ingredients which are suitable according to the invention.

The amount of the abovementioned antioxidants (one or more compounds) in the topical cosmetic compositions according to the invention is preferably 0.001 to 30% by weight, particularly preferably 0.01 to 10% by weight, particularly preferably 0.01 to 5% by weight, based on the total weight of the preparations.

The lipid phase in the topical cosmetic compositions according to the invention comprising 2-hydrazino-1,3-heteroazoles of the general formula I or salts thereof, can advantageously be chosen from the following groups of substances: mineral oils (advantageously paraffin oil), mineral waxes, hydrocarbons (advantageously squalane or squalene), synthetic or semisynthetic triglyceride oils (e.g. triglycerides of capric or caprylic acid), natural oils (e.g. castor oil, olive oil, sunflower oil, soya oil, peanut oil, rapeseed oil, almond oil, palm oil, coconut oil, palm kernel oil, borage seed oil and the like), natural ester oils (e.g. jojoba oil), synthetic ester oils (preferably esters of saturated and/or unsaturated, linear and/or branched alkanecarboxylic acids carrying from 3 to 30 carbon atoms with saturated and/or unsaturated, linear and/or branched alcohols having from 3 to 30 carbon atoms and esters of aromatic carboxylic acids with saturated and/or unsaturated, linear and/or branched alcohols having from 3 to 30 carbon atoms, in particular chosen from the group consisting of isopropyl myristate, isopropyl stearate, isopropyl palmitate, isopropyl oleate, n-butyl stearate, n-hexyl laurate, n-decyl laurate, isooctyl stearate, isononyl stearate, isononyl isononanoate, 2-ethylhexyl palmitate, 2-ethylhexyl laurate, 2-hexyldecyl stearate, 2-octyldecyl palmitate, oleyl oleate, oleyl erucate, erucyl oleate, erucyl erucate, and synthetic or natural mixtures of such esters), fats, waxes and other natural and synthetic fatty substances, preferably esters of fatty alcohols with alcohols of low carbon number (e.g. with isopropanol, propylene glycol or glycerol) or esters of fatty alcohols with alkanoic acids of low carbon number or with fatty acids, alkyl benzoates (e.g. mixtures of n-dodecyl, n-tridecyl, n-tetradecyl and n-pentadecyl benzoate), and cyclic or linear silicone oils (such as, for example, di methylpolysiloxanes, diethylpolysiloxanes, diphenylpolysiloxanes, and mixed forms thereof).

The aqueous phase of the topical cosmetic compositions according to the invention comprising 2-hydrazino-1,3-heteroazoles of the general formula I or salts thereof optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl ether, monoethyl ether or monobutyl ether, diethylene glycol monomethyl or monoethyl ether and analogous products, and also alcohols of low carbon number, e.g. ethanol, isopropanol, 1,2-propanediol, glycerol and in particular one or more thickeners which may advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of Carbopols, in each case individually or in combination, or from the group of polyurethanes.

### EXAMPLES

### Example 1

### “Oil-in-water” Emulsion

| | | | Content |
|---|---|---|---|
| | Raw material name | | in % by |
| Part | (manufacturer) | Chemical name | weight |
| A | Arlatone 983 S ® (ICI) | ether of polyethylene glycol | 1.2 |
| | | with glyceryl monostearate | |
| | Brij 76 ® (ICI) | 3,6,9,12,15,18,21,24,27,30, | 1.2 |
| | | 33,36-decaoxaoctatetracontan- | |
| | | 1-ol | |
| | Cutina MD ® (Henkel) | glyceryl monostearate | 3.5 |
| | Baysiloneöl M10 ® | polydimethylsiloxane | 0.8 |
| | (GE Bayer) | | |
| | Eutanol G ® (Henkel) | octyldodecanol | 3.0 |
| | Paraffin oil 65 cp | mineral oil | 8.0 |
| | (Henry Lamotte) | | |
| B | Water, dist. | | 49.6 |
| | Phenopip ® | 2-phenoxyethanol and methyl | 0.5 |
| | (Nipa Laboratories) | 4-hydroxybenzoate and ethyl | |
| | | 4-hydroxybenzoate and propyl | |
| | | 4-hydroxybenzoate and butyl | |
| | | 4-hydroxybenzoate | |
| | Trilon BD ® (BASF) | disodium EDTA | 0.1 |
| | 1,2-Propylene glycol | | 2.0 |
| | Glycerol 99% | | 3.0 |
| | 2-Hydrazino-1,3-benzo- | | 0.2 |
| | thiazole-5-sulfonic acid | | |
| C | Water, dist. | | 25.0 |
| | Carbopol 2050 ® | crosslinked acrylic acid/ | 0.4 |
| | (B. F. Goodrich) | C₁₀-C₃₀-alkyl acrylate | |
| | | polymer | |
| | Aqueous sodium | | 1.2 |
| | hydroxide solution, 10% | | |
| D | Perfume oil | | 0.3 |

Part A was mixed and heated to 80° C. Part B was mixed and heated to 90° C. and added to part A with stirring. For part C, Carbopol was carefully dispersed in water and neutralized with sodium hydroxide solution (pH 6.9). Part C was then added at 60° C. to the mixture of parts A and B. Part D was added to the mixture of parts A, B and C at room temperature.

### EXAMPLE 2

### “Water-in-oil” Sunscreen Emulsion with UVA/B Broadband Protection

| | | | Content |
|---|---|---|---|
| | Raw material name | | in % by |
| Part | (manufacturer) | Chemical name | weight |
| A | Dehymuls PGPH ® | polyglycerol-2 dipolyhydroxy | 3.0 |
| | (Henkel) | stearate | |
| | Monomuls 90-O 18 ® | glyceryl oleate | 1.0 |
| | (Henkel) | | |
| | Permulgin 2550 ® | beeswax | 1.0 |
| | (Koster Keunen Holland) | | |
| | Myritol 318 ® (Henkel) | caprylic/capric triglycerides | 6.0 |
| | Witconol TN ® (Witco) | C₁₂-C₁₅-alkyl benzoate | 6.0 |
| | Cetiol SN ® (Henkel) | cetyl and stearyl isononanoate | 5.0 |
| | Copherol 1250 ® | tocopherol acetate | 1.0 |
| | (Henkel) | | |
| | Solbrol P ® (Bayer) | propyl-4-hydroxybenzoate | 0.1 |
| | Neo Heliopan ® AV | 2-ethylhexyl | 4.0 |
| | (Haarmann & Reimer) | p-methoxycinnamate | |
| | Neo Heliopan ® E 1000 | isoamyl p-methoxycinnamate | 4.0 |
| | (Haarmann & Reimer) | | |
| | Neo Heliopan ® MBC | 3-(4-methylbenzylidene)-dl- | 2.0 |
| | (Haarmann & Reimer) | camphor | |
| | Neo Heliopan ® OS | 2-ethylhexyl salicylate | 3.0 |
| | (Haarmann & Reimer) | | |
| | Octyltriazone | | 1.0 |
| | Zinc oxide neutral | | 7.0 |
| | (Haarmann & Reimer) | | |
| B | Water, dist. | | 39.8 |
| | Trilon BD ® (BASF) | disodium EDTA | 0.1 |
| | Phenoxyethanol | | 0.7 |
| | Solbrol M (Bayer) | methyl 4-hydroxybenzoate | 0.2 |
| | Glycerol 99% | | 4.0 |
| | Neo Heliopan ® Hydro | 2-phenylbenzimidazole-5- | 10.0 |
| | (Haarmann & Reimer), | sulfonic acid | |
| | 15% as sodium salt | | |
| | Benzophenone-4 | | 0.5 |
| | 2-Hydrazino-1,3-benzo- | | 0.2 |
| | thiazole-5-sulfonic acid | | |
| C | Perfume oil | | 0.3 |
| | Bisabol | | 0.1 |

For part A, all of the substances except zinc oxide were heated to 85° C., and the zinc oxide was carefully dispersed in the mixture. The components of part B were mixed, heated to 85° C. and added to part A with stirring. Part C was added to the mixture of parts A and B and then the mixture was homogenized using a dispersing tool.

### EXAMPLE 3

### “Oil-in-water” Sunscreen Emulsion with UVA/B Broadband Protection

| | | | Content |
|---|---|---|---|
| | Raw material name | | in % by |
| Part | (manufacturer) | Chemical name | weight |
| A | Arlacel 165 ® (ICI) | glyceryl stearate and poly- | 3.0 |
| | | ethylene glycol 100 stearate | |
| | Emulgin B2 ® (Henkel) | ceteareth-20 | 1.0 |
| | Lanette O ® (Henkel) | cetyl and stearyl alcohol | 1.15 |
| | Myritol 318 ® (Henkel) | caprylic/capric triglycerides | 5.0 |
| | Cetiol SN ® (Henkel) | cetyl and stearyl isononanoate | 4.0 |
| | Abil 100 ® | polydimethylsiloxane | 1.0 |
| | (Goldschmidt) | | |
| | Bentone Gel MIO ® | mineral oil and quaternium-18 | 3.0 |
| | (Rheox) | hectorite and propylene | |
| | | carbonate | |
| | Cutina CBS ® (Henkel) | glyceryl stearate and cetyl | 2.0 |
| | | alcohol and stearyl alcohol | |
| | | and cetyl palmitate and | |
| | | cocoglycerides | |
| | Neo Heliopan ® 303 | 2-ethylhexyl 2-cyano-3,3- | 7.0 |
| | (Haarmann & Reimer) | diphenyl-2-propenoate | |
| | Neo Heliopan ® BB | 2-hydroxy-4- | 1.0 |
| | (Haarmann & Reimer) | methoxybenzophenone | |
| | Neo Heliopan ® MA | menthyl anthranilate | 3.0 |
| | (Haarmann & Reimer) | | |
| | 2-Ethylhexyl-N,N- | | 3.0 |
| | dimethyl-4-amino- | | |
| | benzoate | | |
| | Titanium dioxide | | 5.0 |
| | microfine | | |
| B | Water, dist. | | 55.65 |
| | Trilon BD ® (BASF) | disodium EDTA | 0.1 |
| | Veegum ultra ® | magnesium aluminum sulfate | 1.0 |
| | (Vanderbilt) | | |
| | Natrosol 250 HHR | hydroxymethylcellulose | 0.3 |
| | (Aqualon) | | |
| | Glycerol | | 3.0 |
| | Phenopip ® | 2-phenoxyethanol and methyl | 0.3 |
| | (Nipa Laboratories) | 4-hydroxybenzoate and ethyl | |
| | | 4-hydroxybenzoate and propyl | |
| | | 4-hydroxybenzoate and butyl | |
| | | 4-hydroxybenzoate | |
| | 2-Hydrazino-1,3-benzo- | | 0.2 |
| | thiazole-5-sulfonic acid | | |
| C | Perfume oil | | 0.3 |

For part A, all of the substances except titanium dioxide were mixed and heated to 85° C.; the titanium dioxide was carefully dispersed into the mixture. For part B, all of the substances except Veegum and Natrosol were mixed and heated to 90° C., Natrosol and Veegum were dispersed into the mixture, which was added to part A with stirring. Part C was added to the mixture of parts A and B and then the mixture was homogenized using a dispersing tool.

### EXAMPLE 4

### “Oil-in-water” Sunscreen Emulsion with UVA/B Broadband Protection

| | | | Content |
|---|---|---|---|
| | Raw material name | | in % by |
| Part | (manufacturer) | Chemical name | weight |
| A | Crodaphos MCA ® | cetyl phosphate | 1.50 |
| | (Croda) | | |
| | Cutina MD ® (Henkel) | glyceryl stearate | 2.0 |
| | Lanette 16 ® (Henkel) | cetyl alcohol | 1.2 |
| | Myritol 318 ® (Henkel) | caprylic/capric triglycerides | 5.0 |
| | Cetiol SN ® (Henkel) | cetyl and stearyl | 5.0 |
| | | isononanoate | |
| | Copherol 1250 ® (Henkel) | tocopherol acetate | 0.5 |
| | Solbrol P ® (Bayer) | propyl 4-hydroxybenzoate | 0.1 |
| | Abil 100 ® (Goldschmidt) | polydimethylsiloxane | 0.3 |
| | Trilon BD ® (BASF) | disodium EDTA | 0.1 |
| | Neo Heliopan ® HMS | 3,3,5-trimethylcyclohexyl- | 5.0 |
| | (Haarmann & Reimer) | salicylate | |
| | Neo Heliopan ® 357 | butylmethoxydibenzoyl | 2.0 |
| | (Haarmann & Reimer) | methane | |
| B | Water, dist. | | 47.6 |
| | 1,3-Butylene glycol | | 3.0 |
| | Sobrol M ® (Bayer) | methyl 4-hydroxybenzoate | 0.2 |
| | Phenoxyethanol | | 0.7 |
| | Carbopol ETD 2050 ® | acrylic acid/C₁₀-C₃₀-alkyl- | 0.2 |
| | (B. F. Goodrich) | acrylate copolymer | |
| | Keltrol T ® (Calgon) | xanthan gum | 0.2 |
| | Neo Heliopan ® AP | 2,2-(1,4-phenylene)bis(1H- | 22 |
| | (Haarmann & Reimer) | benzimidazole-4,6- | |
| | | disulfonic acid) and | |
| | | disodium salt | |
| | 2-Hydrazino-1,3-benzo- | | 0.2 |
| | thiazole-5-sulfonic acid | | |
| C | Aqueous sodium hydroxide | | 2.8 |
| | solution, 10% | | |
| D | Perfume oil | | 0.3 |
| | Bisabolol | | 0.1 |

Part A was heated to 85° C. Carbopol and Keltrol were dispersed into the remaining constituents whilst cold, the mixture was heated to 85° C. and added to part A. Part C was immediately added at 80° C. to the mixture of parts A and B and homogenized for 5 minutes using a dispersing tool. Part D was finally added at room temperature and the mixture was homogenized using a dispersing tool.

### Comparative Example

The tyrosinase inhibition activity of the exemplary compounds was compared with that of kojic acid as follows:

The tyrosinase enzyme extracted from fungi was obtained from Sigma-Aldrich. The tyrosinase (2000 units/mg) was dissolved in phosphate buffer (pH 6.8, 0.067 mol/1) to a concentration of 120 units/ml, and in each case 100 μl of this tyrosinase solution were introduced into a cavity of a microtitre plate made from polystyrene. 25 μl of phosphate buffer (pH 6.8, 0.067 mol/1) and 75 μl of stepwise-diluted exemplary compound or kojic acid were added. The resulting mixtures were incubated at 37° C. for 10 min. Phosphate buffer (pH 6.8, 0.067 mol/1) was used to dilute the test compounds. The control used was phosphate buffer (pH 6.8, 0.067 mol/1).

100 μl of a 0.03% strength solution of the substrate L-DOPA in phosphate buffer (pH 6.8, 0.067 mol/1) were added, and the absorption (A) was measured at 475 nm using a photometer following incubation for 3 min at 37° C. The residual tyrosinase activities in the presence of Examples 1 to 11 or of kojic acid were calculated in accordance with the following equation:

Residual tyrosinase activity (%)=(A_{Test compound}/A_{control})×100

From the residual tyrosinase activities (%) in a series of dilutions of test compounds, the IC₅₀ was calculated for each test compound. This is the concentration of a test compound at which the tyrosinase is 50% inhibited.

TABLE 1

| | | IC₅₀/ |
|---|---|---|
| Test compound | CAS No. | μM |
| Kojic acid | 501-30-4 | 22 |
| 2-Hydrazino-1,3-benzothiazole | 615-21-4 | 14 |
| 5,6-Dimethoxy-2-hydrazino-1,3-benzothiazole | — | 3.2 |
| 6-Methoxy-2-hydrazino-1,3-benzothiazole | 20174-70-3 | 2.7 |
| 2-Hydrazino-1,3-benzothiazole-5-sulfonic acid | 143269-94-7 | 13 |
| 6-tert-Butyl-2-hydrazino-1,3-benzothiazole | — | 14 |
| 6-Methyl-2-hydrazino-1,3-benzothiazole | 20174-69-0 | 13 |
| 2-Hydrazino-1,3-benzoxazole | 15062-88-1 | 15 |
| 2-Hydrazino-1H-benzimidazole | 15108-18-6 | 70 |
| 2-Hydrazino-5-(4-fluorophenyl)-1,3,4-thiadiazole | — | 12 |
| 2-Hydrazino-4-phenyl-1,3-thiazole | 34176-52-8 | 5 |

1. A topical cosmetic composition comprising 2-hydrazino-1,3-heteroazoles of the general formula 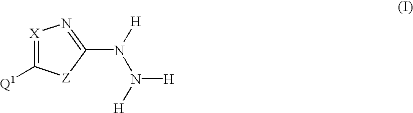
or salts thereof,
where
Z is a sulfur or an oxygen atom or —NH,
and
X is a nitrogen atom or a carbon atom substituted with Q², C—Q²,
and
either
Q¹and Q², independently of one another, are hydrogen atoms, optionally hydroxyl- or alkyloxy-substituted unbranched, branched or cyclic alkyl, alkenyl, 1-oxoalkyl or 1-oxoalkenyl groups having 1 to 18 carbon atoms,
optionally substituted aryl groups having 6 to 15 carbon atoms, optionally substituted heterocyclyl groups having 2 to 15 carbon atoms and at least one heteroatom chosen from the group oxygen, nitrogen or sulfur,
optionally substituted arylalkyl or aryl-1-oxoalkyl groups of 7 to 16 carbon atoms, halogen atoms, nitro groups or groups —COOR⁵, —OR⁵, —NR⁵R⁶, —SO₂OR⁵, —SO₂NR⁵R⁶ or —PO(OR⁵)(OR⁶),
or
Q¹ and Q² together are a radical of the general formula (II) 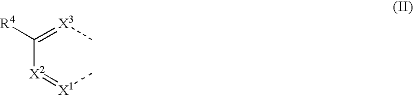
where
X¹, X² and X³, independently of one another, are either nitrogen atoms or
carbon atoms with the radicals R¹, R² or R³, respectively,
and
R¹, R², R³ and R⁴, independently of one another, are hydrogen atoms, optionally hydroxyl- or alkyloxy-substituted unbranched, branched or cyclic alkyl, alkenyl, 1-oxoalkyl or 1-oxoalkenyl groups having 1 to 18 carbon atoms,
optionally substituted aryl groups having 6 to 15 carbon atoms, optionally substituted heterocyclyl groups having 2 to 15 carbon atoms and at least one heteroatom chosen from the group oxygen, nitrogen or sulfur,
optionally substituted arylalkyl or aryl-1-oxoalkyl groups of 7 to 16 carbon atoms, halogen atoms, nitro groups or groups —COOR⁵, —OR⁵, —NR⁵R⁶, —SO₂OR⁵, —SO₂NR⁵R⁶ or
—PO(OR⁵)(OR ⁶)
and
R⁵ and R⁶ independently of one another, are hydrogen atoms, optionally hydroxyl- or alkyloxy-substituted unbranched, branched or cyclic alkyl, alkenyl, 1-oxoalkyl or 1-oxoalkenyl groups having 1 to 18 carbon atoms, optionally substituted aryl groups having 6 to 15 carbon atoms, optionally substituted arylalkyl or aryl-1-oxoalkyl groups of 7 to 16 carbon atoms.

2. A topical cosmetic composition comprising 2-hydrazino-1,3-benzo-heteroazoles of the general formula (I) 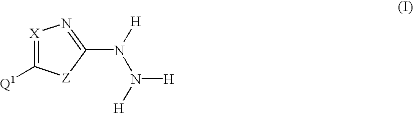
or salts thereof,
where
Z is a sulfur or oxygen atom or —NH,
and
X is a nitrogen atom or a carbon atom substituted by Q², C—Q^{2,}
and
either
Q¹ and Q², independently of one another, are hydrogen atoms, methyl, trifluoromethyl, ethyl, tert-butyl, allyl, 3-methylbut-2-en-1-yl or acetyl groups, chlorine or fluorine atoms, nitro groups, groups —COOR⁵, —OR⁵, —NR⁵R⁶, —SO₂OR⁵, —SO₂NR⁵R⁶, —PO(OR⁵)(OR⁶), phenyl, pyridyl, pyrazinyl groups optionally substituted on the aromatic by alkyl, hydroxyl, alkyloxy, amino, dialkylamino, bromine, fluorine, chlorine, nitrile, sulfonic acid, sulfonamide or alkylsulfonate radicals, phenylmethyl or benzoyl groups,
or
Q¹and Q² together are a radical of the general formula (II) 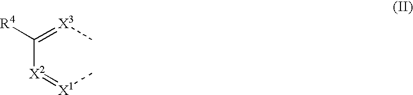
where
X¹, X² and X³ are carbon atoms having the radicals R¹, R² and R³, respectively,
and
R¹, R², R³ and R⁴, independently of one another, are hydrogen atoms, methyl, trifluoromethyl, ethyl, tert-butyl, allyl, 3-methylbut-2-en-1-yl or acetyl groups, phenyl, pyridyl, pyrazinyl groups, phenylmethyl, 4-methyl-phenylmethyl, 4-methoxyphenylmethyl or benzoyl groups, chlorine or fluorine atoms, nitro groups, groups —COOR⁵, —OR⁵, —NR⁵R⁶, —SO₂OR⁵, —SO₂NR⁵R⁶ or —PO(OR⁵)(OR⁶)
and
R⁵ and R⁶, independently of one another, are hydrogen atoms, methyl, trifluoromethyl, ethyl, tert-butyl, allyl, 3-methylbut-2-en-1-yl, acetyl, propionyl or pivaloyl groups, phenyl, pyridyl, pyrazinyl groups, phenylmethyl, 4-methylphenylmethyl, 4-methoxyphenylmethyl or benzoyl groups.

3. The topical cosmetic composition, in particular a cosmetic or dermatological skin-lightening composition, as claimed in claims 1 and 2, comprising
2-hydrazino-1,3-benzothiazole
5,6-dimethoxy-2-hydrazino-1,3-benzothiazole
6-methoxy-2-hydrazino-1,3-benzothiazole
2-hydrazino-1,3-benzothiazole-5-sulfonic acid
2-hydrazino-1,3-benzothiazole-6-sulfonic acid
6-tert-butyl-2-hydrazino-1,3-benzothiazole
6-methyl-2-hydrazino-1,3-benzothiazole
2-hydrazinothiazolo[5,4-b]pyridine
2-hydrazino-1,3-benzoxazole
2-hydrazino-1H-benzimidazole
2-hydrazino-5-(4-fluorophenyl)-1,3,4-thiadiazole
2-hydrazino-4-phenyl-1,3-thiazole
2-hydrazino-4-methyl-1,3-thiazole hydrochloride.

4. The topical cosmetic composition as claimed in claims 1 to 3, comprising
0.001% by weight to 30% by weight of the 2-hydrazino-1,3-heteroazoles, based on the total weight of the formulation.

5. The use of the 2-hydrazino-1,3-heteroazoles as claimed in claim 1 to 4 in cosmetic preparations.

6. The use of the 2-hydrazino-1,3-heteroazoles as claimed in claim 1 to 4 in dermatological preparations.

7. The use of the 2-hydrazino-1,3-heteroazoles as claimed in claim 1 to 4 in cosmetic or dermatological skin-lightening compositions.

8. The use of the 2-hydrazino-1,3-heteroazoles as claimed in claim 1 to 4 in sunscreens.

9. The use of the 2-hydrazino-1,3-heteroazoles as claimed in claim 1 to 4 in hair care products.

10. The use of the 2-hydrazino-1,3-heteroazoles as claimed in claim 1 to 4 in anti-skin-aging products.

11. The use of the 2-hydrazino-1,3-heteroazoles as claimed in claim 1 to 4 in combination with other skin lightening active ingredients or active ingredient mixtures in topical cosmetic compositions, in particular cosmetic or dermatological skin-lightening compositions.

12. The use of the 2-hydrazino-1,3-heteroazoles as claimed in claim 1 to 4 in combination with antioxidative or free-radical-scavenging active ingredients or active ingredient mixtures in topical cosmetic compositions, in particular cosmetic or dermatological skin-lightening compositions.
